(19) United States
(12) Patent Application Publication
(10) Pub. No.: US 2019/0200577 A1
(43) Pub. Date: Jul. 4, 2019

(54) DOG HARNESS FOR HEALTH DATA COLLECTION AND MONITORING

(71) Applicant: Liy Kath, San Francisco, CA (US)

(72) Inventor: Liy Kath, San Francisco, CA (US)

(21) Appl. No.: 15/859,330

(22) Filed: Dec. 30, 2017

(51) Int. Cl. A01K 27/00; A01K 29/00; A61D 13/00; A61B 5/0205; A61B 5/00

(52) U.S. Cl. CPC A01K 27/009; A01K 27/002; A01K 29/005; A61D 13/00; A61B 5/02055; A61B 5/6831; A61B 5/02438; A61B 5/743; A61B 5/7435; A61B 5/7445; A61B 5/746; A61B 5/0022; A61B 5/7282; A61B 5/7425

(57) ABSTRACT

A dog harness for health data collection and sensor-based vital sign and activity level measurement and display. The harness provides health data monitoring with integrated temperature, heart rate and breathing rate sensors and transducers, pedometer, and GPS for measuring activity levels, and a harness mounted, programmable, interactive, touch controlled, flat panel display for viewing and interacting with the dog's health data. Health data, notifications, status updates, messages, reminders, and warnings are displayed graphically or numerically on the harness integrated LED or flat panel display screen. Health data is continuously uploaded to cloud-based services for processing, analysis and diagnosis. Software applications display health data on a mobile device or desktop computer. The flat panel display is a narrow form factor design mounted near the dog's neck on the top of the harness. The harness distributes the leash pulling forces away from the neck towards the dog's center of mass.

### BACKGROUND

Monitoring the health, vital signs, and activity level of a pet dog is useful for the owner as caregiver and beneficial to the companion animal's well-being. Understanding and monitoring a dog's body temperature, heart rate and breathing rate provides health data from which fluctuations and deviations from normal physiological states is useful for maintaining the animal's proper physical and mental health. Collecting and monitoring a dog's temperature, heart rate, breathing rate, audible signals, and activity levels from harness-based electronic sensors is an advantageous method for observing health state and vital signs. Health status information and vital signs may be streamed and displayed across a harness-mounted LED screen to provide instantaneous access to real-time health data. Health data readings, current body temperature, current heart rate, current breathing rate, status updates, reminders and warnings may be generated and displayed on the harness-mounted LED screen for viewing by the owner.

Uploading the measured vital signs, activity levels and collected health data to cloud based storage allows the dog owner and veterinary professionals to interpret and track the dog's current state of health and see health trends over time. Cloud-based or remotely-hosted system software and algorithms may process and model the dog's uploaded health data. Proper actions may be taken during automated system generated health warning in the form of emergency care, provision of water and food, or other healthcare procedures. Additionally, adjustments in activity levels and diet may be made in order to improve the animal's current health state and overall lifestyle. Software applications and wireless hardware are used to collect health data from harness based sensors and upload the data to cloud-based or remotely hosted health data processing services. The dog owner or veterinarian may download available raw health data or automatically generated system reports to mobile devices and web-based desktop computer applications for interactive viewing, statistical modeling, and diagnosis.

The technology product disclosed here may be marketed as “The Betta Noir K9i” dog harness manufactured and sold by Yap USA Inc. The dog harness uses a plurality of sensor probes and electronic modules to measure temperature, heart rate, breathing rate, and activity levels. The harness mounted sensor probes may be wired and positioned inside the harness to contact the dog's chest, legs, back or neck area. Sensor probes are specifically designed to penetrate the dog's hair and make contact with the skin in order to provide accurate and consistent measurements and signal data. Redundancy though multiple probe sensing apparatus is designed into the sensor measurement system to ensure uninterrupted data collection and monitoring. For example, a preferred embodiment of the presently disclosed dog harness may utilize three sensor probes for temperature inside the chest area of the harness. A fourth sensor probe may additionally be mounted in the neck area of the dog harness for further redundant temperature sensor signal and data collection. The use of a plurality of temperature sensors ensures an uninterrupted stream of body temperature data to the LED screen and data upload to cloud-based services. In situations where the dog's movement causes a temperature sensor to lose contact with the skin or otherwise be unable to measure body temperature, the other redundant temperature sensors in the harness will be able to maintain continuous updates to the harness system computer with sensor signals and data.

Conventional and currently available dog collars, choke chains, and harnesses pull at a dog's neck and can cause air passage, tracheal, or laryngeal damage. Furthermore, when a dog is pulled from the neck, the dog's equilibrium is affected and tremendous wear and tear is put on the hip joints as the dog attempts to counteract the disruptive force. The presently disclosed harness system provides for moving the center of gravity of the leash pulling force towards the torso and stomach of the dog. The harness system here moves the forces encountered by the dog from the leash towards the dog's center of mass. The harness provides for an even distribution of force and the avoidance of long-term injury caused by forces on the hips, potential hip dysplasia, and neck injury. The harness also facilitates improved communication by the owner and listening by the dog through better control by evenly distributing the tugging, pulling, and guiding forces applied through the owner's manipulation of the leash attached to the harness.

Guide dogs, service dogs, and work dogs with critical dependability status for their owners will be aided by the dog harness system disclosed here. Dog health data and vital signs may be output to accessibility tailored interfaces depending on the situation or use case scenario. As an example, a service dog for people with visual impairment may provide health data updates via a touch-based interface incorporated into the dog harness. In another embodiment of the harness system interface, the health data updates may be provided via audible cues or notifications. The harness system interface can signal audible health data updates through voice activated command and control. Alternatively, the service dog may operate and interface with a harness-based sensor system which measures, collects and uploads health data directly to veterinary providers for assistance and care without needing or requiring action on the part of the dog owner. The veterinary provider or care specialist may download the health care data from cloud-based services and process the information for diagnosis and determination of actionable treatment options. Health data reports, notifications, and emergency warnings may be generated by cloud-based software and transmitted to the appropriate dog health care actors or stakeholders for proper care procedures.

The health data, vital signs and dog activity level measured and collected by the dog harness is uploaded to cloud based databases for data analytics and processing. The cloud based software system provides for the revolutionary analysis of aggregate customer health data. Product improvements to harness and sensor design may be determined from analyzing across the entire population of customer data. Dog related healthcare issues, diseases and treatment best practices may be realized and developed through statistical modeling of the health data population, vital sign statistical analysis, and activity level monitoring for certain breeds or across the entire customer dog population. For example, cloud based analytics of dog health data may reveal trends and provide helpful diagnosis to the actual causes of pain issues in specific areas, muscles, or joints of the customer's dogs. The health care data analytics may aid in the determination of specific physiological impact or injury from measured activity conditions.

### SUMMARY

A dog harness with sensor-based vital sign measurement of temperature, heart rate, breathing rate and activity level is provided for real-time display via harness-mounted LED screen and upload to cloud based services for health data collection, modeling and diagnosis. The harness includes a plurality of electronic sensors, modules and computer processing units for redundancy and uninterrupted heath data and vital signs data acquisition from the dog. The harness-mounted LED screen provides for and easily readable and understandable interface to view and interact with health data for the dog's owner or veterinarian. Health data and vital signs information is wirelessly uploaded to cloud services to allow display on a connected mobile device or desktop computer. Server and cloud-based analytics and data modeling may be applied to a customer dog's health data for the generation of automated reports and status updates regarding product usage, dog health diagnosis, and emergency care. Dog health care data may be transmitted to the dog's owner, veterinary provider, or caretaker specialist for the determination of actionable dog health care procedures.

### DESCRIPTION OF THE DRAWINGS

FIG. 1 is a view of a dog wearing the harness system for health data collection and monitoring with sensor-based vital sign and activity level measurement and display.

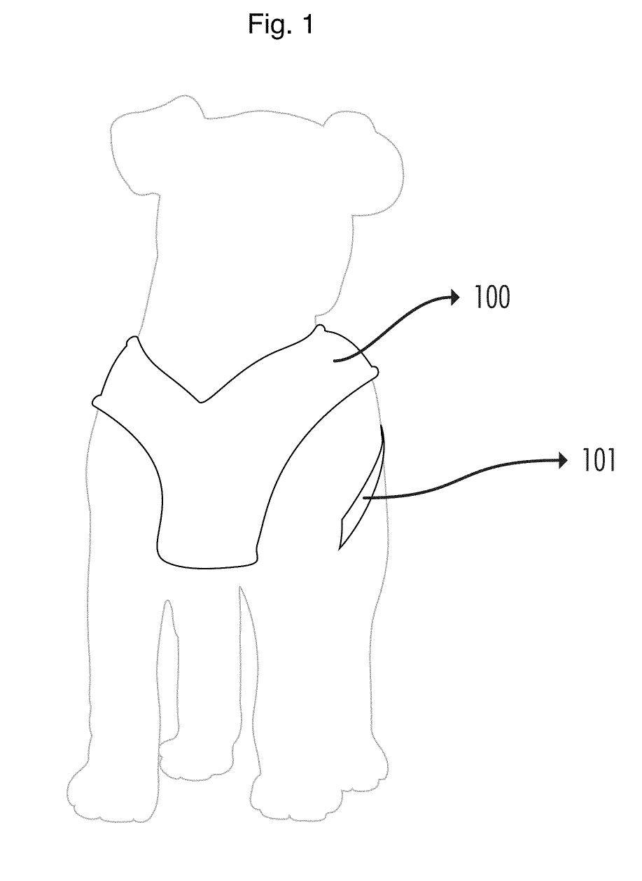

FIG. 2 is another view of a dog wearing the harness system for health data collection and monitoring with sensor-based vital sign and activity level measurement and display.

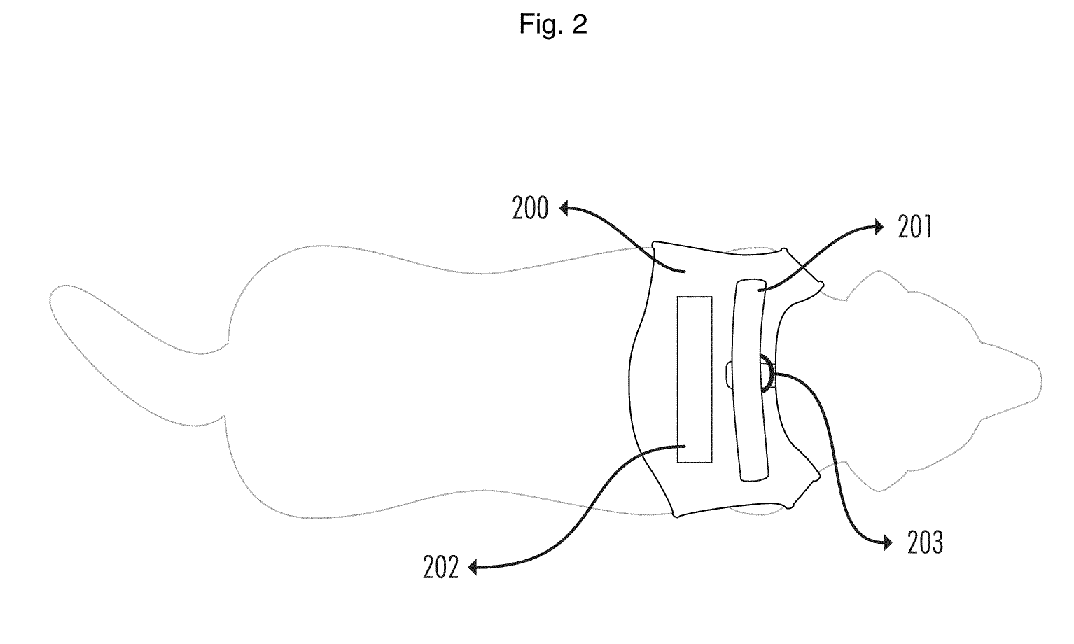

FIG. 3 is a view of the programmable, interactive and manipulative, touch controlled, harness-mounted LED screen, or flat panel display, for providing health data readings, body temperature, breathing rate, heart rate, status updates, messages, reminders and warnings.

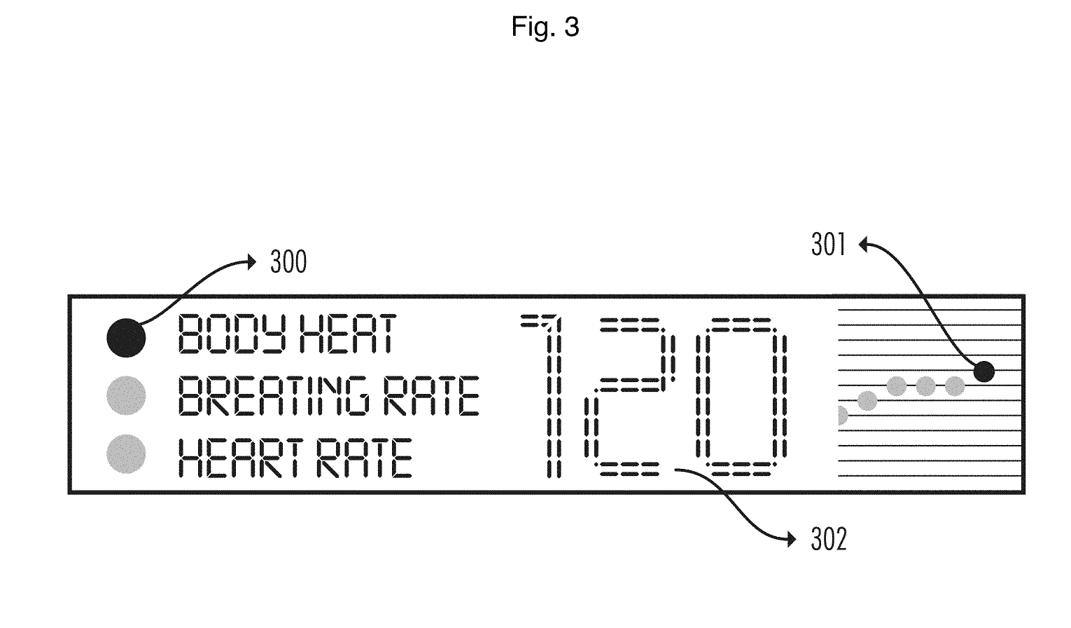

FIG. 4 is an alternate view of the harness-mounted LED screen, or flat panel display, for providing health data readings, body temperature, breathing rate, current heart rate, status updates, messages, reminders and warnings.

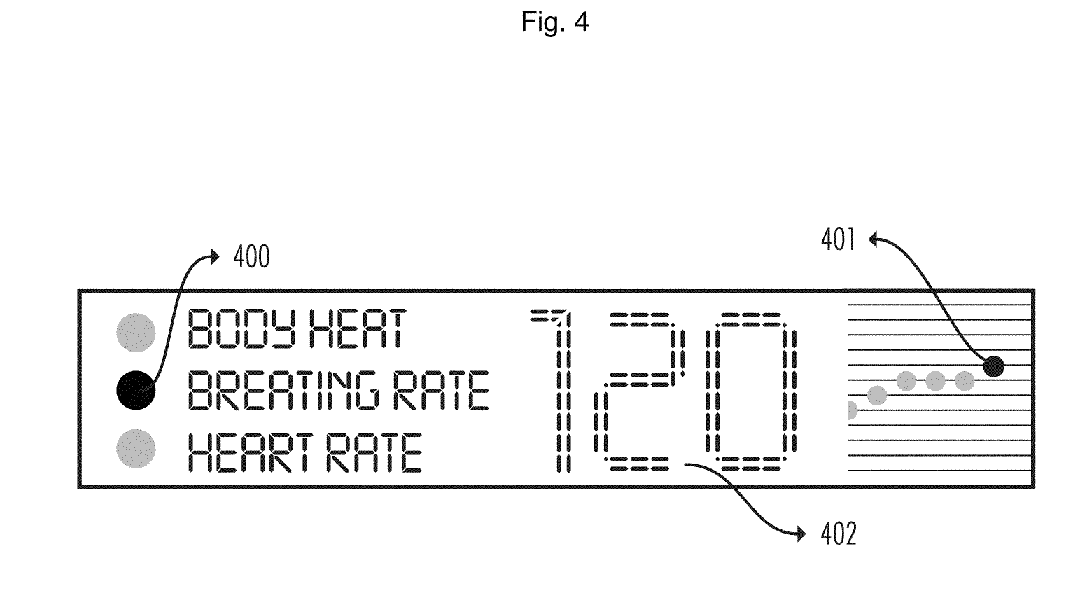

FIG. 5 is an additional alternate view of the harness-mounted LED screen, or flat panel display, for providing health data readings, body temperature, breathing rate, heart rate, status updates, messages, reminders and warnings.

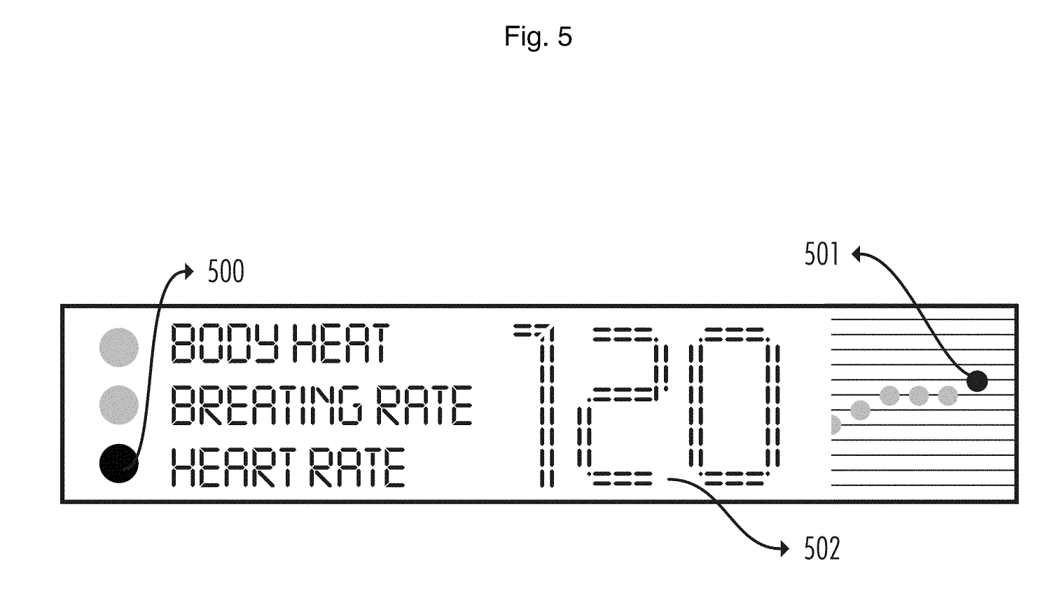

FIG. 6 is a view of the plurality of sensor probes integrated into the harness to provide accurate and consistent measurements and signal data, health data readings, body temperature, breathing rate, and heart rate.

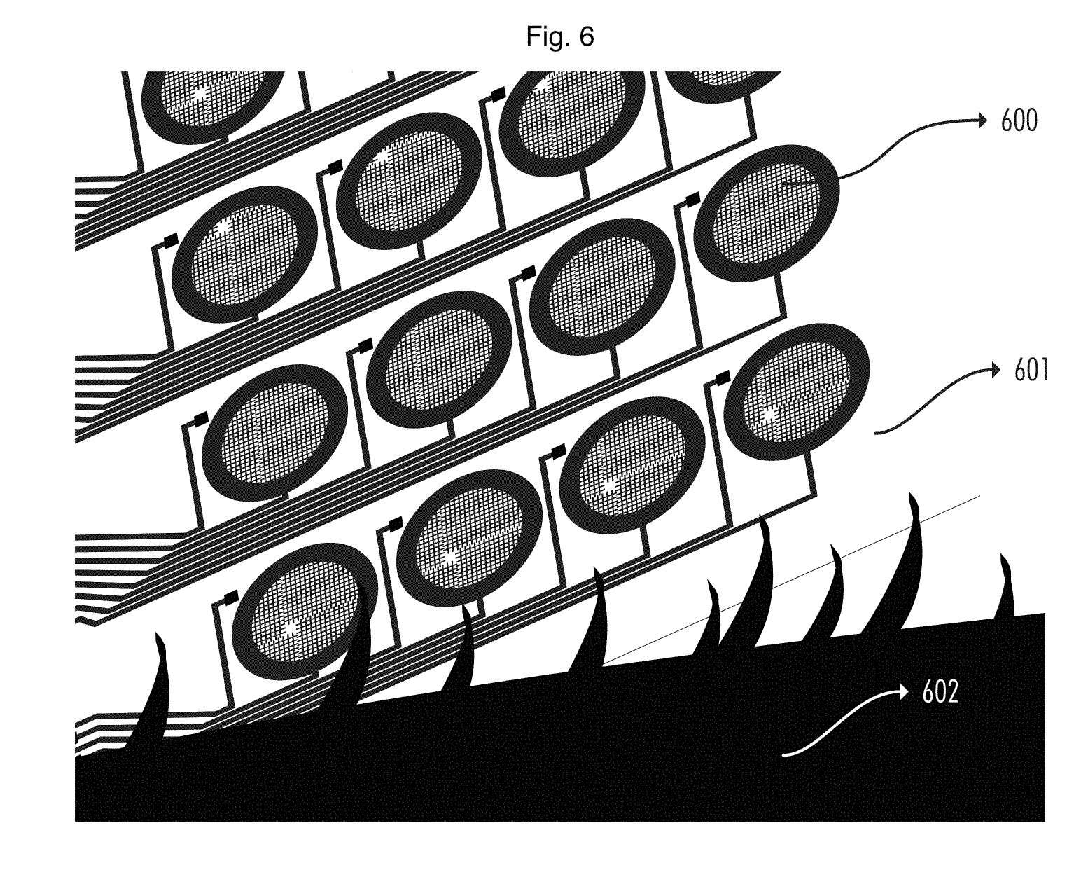

FIG. 7 is a close-up view of the dog harness system for health data collection and monitoring and the integrated harness mounted LED or flat panel display screen for providing health data readings and vital sign information, showing the narrow dog collar sized form factor design mounted near the dog's neck on top of the harness.

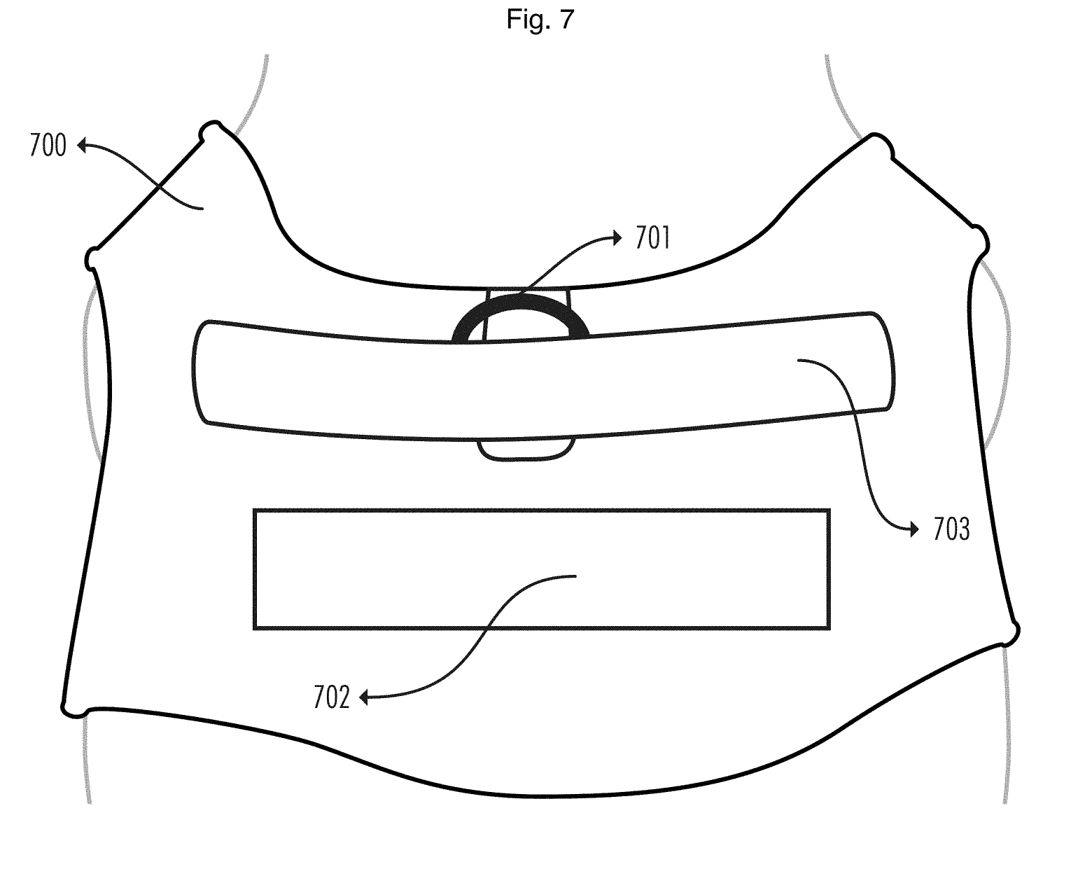

### DETAILED DESCRIPTION

A dog's health data may be acquired, displayed and interpreted with the presently disclosed dog harness system through the measurement of vital sign information (i.e., body temperature, heart rate or pulse, and breathing rate) and activity levels with specifically engineered harness mounted sensing circuitry. The dog's body temperature, heart rate, breathing rate, audible signals and activity levels are measured with the sensor probes, microphones and related electronic circuitry and modules wired into the harness, displayed on a harness mounted LED screen and wirelessly uploaded to cloud based services for accessibility and interpretation with mobile devices and desktop computer applications.

The acquisition of a dog's body temperature by the harness system is accomplished with the placement of a plurality of temperature sensors within the harness. An embodiment of the presently disclosed harness system may incorporate three chest mounted and one neck or back mounted temperature sensor for a total of four sensors. Alternative numbers and placements of temperature sensors may be employed within the harness system to ensure accurate signal data acquisition and un-interruption. Redundancy in temperature sensor placement is advantageous for uninterrupted signal data as various sensors in the system may be subject to mechanical or electromagnetic interference, or may lose and re-acquire signal during sustained motion and movement of the dog while coupled to the harness. During normal daily activity, the dog's movement may cause temperature sensors in the harness system to lose contact with the dog's skin, become inactive, or otherwise lose the ability to accurately measure body heat signals. The use of a plurality of redundant temperature sensors in the system ensures a constant supply of uninterrupted data streams to the harness system computer for collection and monitoring.

The harness system body heat and temperature sensor probes are specifically engineered to penetrate the dog's hair and make viable contact with the dog's skin in order to provide accurate measurements. Sensor probe contact with the skin is desirable to obtain accurate measurement and collection of body temperature data. Therefore it is advantageous to design the harness sensor probe mechanism to produce positive force towards the dog's body and sustain contact through the hair. The temperature sensor probe tip disclosed here is designed with a bendable rounded metallic tip that penetrates the dog's hair and contacts the skin. The probe tip may be metallic and rounded in construction with spring force produced tension for penetration through the dog's hair. The sensor probe may be mounted in a plastic frame housing the sensor probe tip, a force plate, retaining rings, and a spring force tensioner producing a positive downward force on the sensor probe tip. The sensor probe frame housing may be circular in shape and ¼, ⅜, or ½ inches in diameter. The spring force tensioner may produce a positive force of 0.5 lbs. or greater up to 10 lbs., depending on the sizing for the specific harness application and desired adjustment. The dampening and spring force may be adjusted on the sensor probe frame housing with a thumbscrew adjustment. Alternatively, the sensor probe tension, contact, and applied force may be automatically adjustable by the harness electronic modules.

Another embodiment of the temperature probe in the harness system may be designed with a rounded metallic tip for penetration through the dog's hair by computer actuated servo-motors which turn in a radial or axial motion to drive the probe tip down through the dog's hair. The servo-motor and sensor probe apparatus may be mounted in a plastic frame near the harness chest area. When the harness is affixed to the dog's chest, the computer controlled servo-motor will actuate a radial turning motion of the sensor probe tip within the plastic frame. The sensor probe will begin traveling downward through the dog's hair until the appropriate force and contact with the skin is achieved for accurate and sustained body heat temperature data is acquired by the harness computer. Alternatively, the serve motor control mechanism may turn the sensor probe in an axial motion to drive the sensor probe tip downward towards the dog's skin and produce enough force to penetrate the hair. The servo motor drive will be calibrated to produce only enough force that is necessary to achieve the proper contact with the dog's skin by the sensor probes. Consideration will be made to not produce harmful or uncomfortable downward force on the dog's skin. The acquisition by the harness system computer of reliable body temperature sensor data will feedback to the computer controlled servo motor drive and regulate the appropriate force in the system. For example, once the servo motor system achieves sensor probe contact with the skin, the temperature data will be received by the harness system computer, and the servo motor will discontinue adjustment in the radial or axial motions.

An alternative embodiment of the temperature probe may be designed as a rounded metallic tip coupled to a manual harness mounted and operated twist-adjustment mechanism for placement and penetration of the probe through the dog's hair. The manual twist adjustment may be activated by hand turning the adjustment knob on the exterior of the harness. The rotation of the adjustment knob will drive the sensor probe tip downward through the dog's hair in order to make viable contact with the skin. The manual twist mechanism is built into a plastic frame housing, with the sensor probe tip placed below a screw knob adjustment. The mechanism is built into the harness system near the chest area of the dog.

Another alternative embodiment of the temperature probe may be a metallic tip based probe with depth adjustment provided by harness mounted hand-operated manipulation for penetration through the dog's hair for viable contact with the skin. The sensor probe tip is manipulated with a knob and fixture based housing, preferably constructed out of plastic or nylon with a sliding groove. The hand operated knob faces outside the harness and manipulates the sensor tip inside the harness and near the dog's chest and torso area. The knob may be manipulated and slid inside the grove of the fixture housing in order to adjust the downward tension and force onto the dog's hair and skin. Ultimately, the proper adjustment can be found through the acquisition of body heat and temperature sensor data by the harness system computer.

Another embodiment of the temperature probe may be a rounded metallic tip with harness strap activated and adjustable tension which provides secure contact to a hairless part of the owner's dog. It is understood that certain use case scenarios may require the owner to shave or trim the dog's hair in the area of the temperature sensor probe contact. Certain critical and high availability uses may require the modification of the dog's hair to suit the owner's requirements for temperature and body heat data acquisition. For example, a service dog in a zero risk tolerance and extreme dependability situation, as a rescue dog or guide dog for the visually impaired, may require the removal of hair in the area of the temperature sensor probes for uninterrupted and reliable body heat data collection. The harness mounted strap mechanism for the sensor probe allows adjustment and secure downward force of the sensor probe tip to the hairless part of the dog's chest or torso. Contact with the dog's hairless skin provides accurate and uninterrupted collection of temperature data.

Another alternative embodiment of the temperature probe may comprise a rounded metallic tip probe wired into a spring-tensioned probe arm which is ergonomically designed to penetrate the dog's hair and automatically make contact with the skin upon proper securing and adjustment of the harness system to the dog. An ergonomically positioned and tensioned sensor probe arm is built into the chest area of the harness. Upon securing the harness system to the dog, the ergonomically designed sensor probe is pushed into the dog's chest and through the hair. Successful contact with the skin is achieved by the sensor probe tips. Positive contact is sustained though the ergonomics of the sensor probe tip arm, the harness, and the positive force from secure harness adjustment. As a preferred embodiment of the ergonomically positioned sensor probes, the probes are aligned along the chest facing area of the harness. Positive tension of the sensor probes is produced by plastic ribbing embedded inside the harness system in the chest area. The plastic ribbing is curved and angled in an convex or concave manner to product positive force towards the dog's chest and force the sensor probe tips down through the hair and onto the dog's skin. The adjustment of the harness presses the sensor probe tips against the dog's chest. Furthermore, tension against the chest is maintained by the plastic ribbing. Further embodiments of the harness system temperature sensor apparatus and sensor probes may utilize combinations of all or some of the above described methods.

Redundancy in temperature sensor probes in the harness system will also be used to improve signal and data quality and integrity. The dog harness system and computer may collect and stream temperature data from four probes simultaneously in a preferred embodiment of the invention. Temperature signal data may be processed by the harness system on-board computer and averaged or algorithmically processed for data reliability. Temperature probe signal noise can be reduced through the direct comparison of raw signal data between alternate sensor probes in the harness. The harness system's on-board computer can acquire raw data from two different temperature sensor probes and use algorithms for the reduction and elimination of signal data noise. Temperature data anomalies can be taken out and removed from the sampling data by specifically formulated comparison algorithms and processing by the harness based computer.

The temperature sensor probes in the harness system are hard wired to the sensor driving, amplification and filtering circuitry and electronic modules which are in turn hard wired and connected to the on-board computer. Sensor signal data is acquired by driving and powering the sensing hardware, detecting the signal, amplifying the raw signal and filtering for acquisition by the harness computer. A preferred embodiment of the sensor probe wiring is by harness embedded and protected wiring connecting the temperature probe tips to the sensor driving, amplification, and filtering circuitry and on-board computer. The wiring may be covered in a rubber or other waterproof sheath to guard against moisture and corrosion. Additionally, the wiring may also be embedded inside a fabric sheath to guard against mechanical wear and tear and interference.

The acquisition of a dog's heart rate or pulse by the harness system is accomplished by the placement of an array of acoustic sensors within the chest area of the harness so as to be near the dog's heart or chest cavity. A preferred embodiment of the presently disclosed harness system may incorporate three acoustic sensors mounted between the arms and on the chest area of the dog. Alternative embodiments of the harness system may utilize various numbers of acoustic sensors or sensor arrays to ensure accurate and uninterrupted heart rate signal data. Redundant placement of acoustic sensors in the harness near the dog's chest cavity is advantageous for high-quality sensor signal data and for avoiding interruptions is measurement.

In an alternative embodiment of heart rate or pulse measurement by the harness system, the sensors may be incorporated into an arm band to be placed around the dog's front limb or bicep area. The arm band will contain sensors or a sensor array for measuring the dog's heart rate or pulse at the arm. The acoustic sensors will have wired connections feeding back through the main harness and to the harness system computer. The arm band will be secured with a Velcro strap and stretchable webbing strap with plastic or nylon clips or buckles.

The dog's heart rate or pulse signal data from the sensor array is received by the harness system computer for processing and adjustment for accuracy. The heart rate or pulse acoustic signal is detected by the sensors and computer and the signal is further filtered and amplified. Acoustic sensor signal noise can be reduced and eliminated through comparison and averaging of sensor signal data streams across the array. The harness system computer can apply statistical modeling and specific algorithms to the acoustic signal streams to remove signal noise and data anomalies from the raw sampling data.

The sensor array in the harness system is hard wired to the sensor driving, amplification and filtering circuitry and modules located within the harness and near the on-board computer. Sensor signal data is acquired by driving and powering the acoustic sensor array hardware, detecting the signal, amplifying the raw signal and filtering signal noise for acquisition and measurement by the harness computer. The sensors that comprise the array may be embodied by electro-mechanical transducers that are sensitive to the sound wave energy levels of a dog's heartbeat. Alternatively the sensor array may be designed to measure the electrocardiogram (EKG) signal from the dog's heart muscles and display this data on the harness system interface as a raw EKG signal graph or covert the signal to heart rate or pulse numerical data. Electrodes will be used to acquire the EKG signal data from the dog's chest or torso area.

The acquisition of a dog's breathing rate by the harness system is accomplished by the placement of an array of acoustic sensors within the harness and near the lungs of the dog. An embodiment of the presently disclosed harness system may incorporate an array of three acoustic signals mounted on the chest of the harness and near the dog's lung cavities. Alternative numbers and placements of the breathing rate acoustic sensors may be employed within the harness system to ensure accurate signal acquisition and un-interruption. Redundancy in breathing rate acoustic sensor placement is advantageous for uninterrupted signal data as signal noise and inability to acquire data may occur due to sustained motion and movement of the dog.

The dog's breathing rate signal data from the acoustic sensor array is received by the harness system computer for processing and error correction. The breathing rate acoustic signal is detected by the sensor array and processed by circuitry dedicated to filtering and amplification for the harness computer. Acoustic sensor signal data is acquired by the harness computer by driving and powering the acoustic sensor array hardware, detecting the signal, amplifying the raw signal and filtering for noise. The acoustic sensors comprising the breathing rate sensor array are embodied by electro-mechanical transducers that are specifically attuned to the sound wave characteristics and energy levels of a dog's lungs.

The acquisition of a dog's activity level by the harness system may be accomplished by vibration detection, accelerometer, gyroscope, compass, pedometer, or GPS device incorporated into the harness. An embodiment of the activity level measurement in the harness system may comprise an electronic module with an accelerometer, gyroscope and compass for determining the amount and rate of activity performed by the dog in a given period of time. Another embodiment of activity level measurement may be comprised of a pedometer unit embedded within the harness which acts to count steps taken by the dog. In another embodiment of the activity level measurement functionality, GPS antennae and electronics may be incorporated into the harness system for receiving GPS signal coordinates and logging distance traveled by the dog. The distance traveled by the dog may be calculated by the on-board computer and available for display on the harness mounted LED screen. Activity level status may be displayed by the LED screen as ACTIVE, INACTIVE, or NORMAL along with historical activity levels for the owner's dog and related data for comparison of particular dog breed normal baseline activity levels depending on and configurable to reflect the time of day, age, or breed.

Health data display functionality may be embodied in a programmable, interactive and manipulative touch controlled harness mounted LED (light-emitting diode) screen, OLED (organic light emitting diode), LCD screen (liquid-crystal display), or other flat panel display with designated status messages and menu display features for quickly readable and easily understandable health status updates and notifications. A preferred embodiment of the LED screen may be a narrow dog collar sized form factor design near the dog's neck on the top of the harness. The intention of the LED screen is to provide health data readouts and displays so that the dog owner can at any time read and see health data, vital signs, and activity levels of the dog. For example, while wearing the harness, the body temperature of the dog may be displayed in an easily understandable natural color scheme to signify heat levels or temperature zones, i.e., GREEN for healthy or normal, YELLOW to indicate a deviation from normal dog body temperature, or RED for a clear warning of a severe problem with body temperature. The overall intention of the LED screen updates are a quick assessment of the animal's health condition by the owner through simple visual cues.

The heart rate signal is displayed on the LED screen in a heart rate or heart beat menu display. The owner may select on the LED screen the heart beat signal from available options. The dog's currently measured heart beat signal graph can be displayed on the LED screen as an electro-cardiogram or EKG graph. Also the dog's numerical heart rate (HR) or pulse is displayed and available for viewing in the menu selection functionality. The dog owner may select from the menu on the LED screen to view the heart rate data and will be provided with current pulse or beats per minute (BPM), and high or low BPM for historical levels associated with the particular dog. Status updates and notifications can be displayed to inform the dog owner of normal or non-normal dog heart rate levels. For example, a RED warning message may alert the dog owner when the dog's heart rate is at an elevated level. Similarly, the breathing rate or respiratory rate (RR) is displayed on the LED screen as breaths per minute. High or low respiratory rates for historical levels may be displayed on the screen, as well as warning messages for abnormal respiratory rates. Additionally, status identifiers such as OK, NORMAL, WARNING, ELEVATED, or BELOW NORMAL may be displayed to give the dog owner easily understandable health data diagnosis regarding body temperature, heart rate or respiratory rate.

The LED screen, or flat panel display, will provide a user-interface (UX) design and functionality tailored for allowing health data and vital sign information to be readily interpreted and accessed by the dog owner or caregiver. A preferred embodiment of the user-interface design for the dog's temperature or body heat level may comprise the following. Initially the harness system is attached and secured to the dog and turned on and temperature data begins to be acquired and streamed by the temperature sensor probes to the harness system computer. While in temperature mode, the LED screen will flash “TEMP PROBE OK” to indicate that the temperature sensor probes are functioning correctly, have proper contact with the dog's skin, and are streaming body heat data to the harness system computer. If none of the temperature sensor probes are able to acquire data, by either non-contact with the dog's skin or other malfunction, the LED screen will alert the user with the message “TEMP PROBE RE-ADJUST” or “NO TEMP DATA” and display a graphic or pictorial notification to alert the dog owner to re-position the harness and temperature probe array. Additionally, an audible warning may be given to alert the owner that the temperature sensor probe data is not being acquired. With proper temperature sensor probe data being acquired by the harness system computer, the LED screen will display the temperature as numerical data, such as 101° F., or 102.5° F. (or 39.2° C.). Normal temperature ranges will be given in GREEN to alert the dog owner of proper healthy physiological levels. The temperature reading may be displayed constantly, flashed periodically at set intervals, for example every 30 seconds, or alternatively may be displayed at every change in temperature. During increased activity levels, the dog's temperature will rise and the temperature sensor probe data will be streamed to the computer and displayed on the LED screen during rising intervals. Typically if the temperature rises above 104° F. a warning message will be given across the LED screen as “WARNING ELEVATED” and this may be flashed in RED for a clear warning message. Alternatively, if the temperature falls below 99° F. a warning message will be given as “WARNING BELOW NORMAL”. Audible warnings may be given during high or low temperature events. Additionally, during swings or changes in temperature levels out of normal physiological ranges, the temperature data may be flashed across the LED screen in YELLOW color to indicate a transition out of the safe and normal green zone and into potential serious hyperthermic or hypothermic conditions, and audible signals may be given to indicate changes.

The harness system LED screen, or flat panel display, may be switched to heart rate mode or menu functionality by touching a menu button near the screen or by interacting with the screen's touch-sensitive functions. In the heart rate menu, the dog's acoustic sensor array data will be displayed to indicate current heart rate or pulse levels. In a preferred embodiment of the user-interface (UX), the LED screen will flash “HEART RATE SENSOR OK” to indicate the proper measurement and streaming of heart rate signals by the acoustic sensors in either the chest mounted sensor array or the arm-band sensors. If the heart rate sensors are not able to acquire heart rate signal data, the screen will display “HEART RATE SENSOR RE-ADJUST” to alert the dog owner that the sensors require adjustment or alignment in the harness chest area or arm band. Upon the successful measurement and collection of heart rate signal data by the harness system computer, the LED screen may display the heart rate (HR) numerically as “60, 70, 80, 90, 100, 120, or 140 BPM” (beats per minute), depending on the actually measured data over a pre-determined time period interval. Additionally, the heart rate data may be displayed on the LED screen in colors to indicate normal levels, changes out of normal ranges, and high or low warning levels. In a preferred embodiment of the heart rate user-interface (UX), the acoustic sensor signal data is displayed numerically as “70 BPM” in GREEN color to indicate the dog's current heart rate and that the level is physiologically normal and healthy. The heart rate numerical data may be constantly displayed, flashed periodically during set 30 second intervals, or alternatively flashed upon the LED screen during swings or changes out of current levels. During activity, the dog's heart rate may rise and the LED screen will flash updates in YELLOW to indicate a rise in heart rate level, i.e., “80, 90, 100 BPM” may be displayed on the screen and audible alerts may be given to indicate changes in heart rate. If the dog's heart rate levels rise or fall out of normal levels, the LED screen will display the measured heart rate in RED color and an audible warning signal may also be given. In another preferred embodiment of the LED screen user-interface (UX) for heart rate menu functionality, the dog owner may select EKG (electrocardiogram) mode and see the dog's measured EKG signal. The EKG signal will then be displayed on the screen as a graph showing the dog's heart muscle activity as measured from harness mounted electrodes contacting the dog's skin. Audible EKG signals may also be given from the harness system to provide another mode of receiving information.

The harness system LED screen, or flat panel display, will also have a breathing rate mode display or menu functionality. By activating the menu button or touch screen functionality on the LED screen, the dog owner may navigate through the menus to display the breathing rate menu display. During successful acquisition and streaming of breathing rate sensor data to the harness system computer, the LED screen will display “BREATHING RATE SENSOR OK”. During the unsuccessful acquisition of breathing rate data, an alert or other notification will be given to provide notice to the dog owner that the breathing rate signals are not being acquired by the harness system computer, for example as “BREATHING RATE SENSOR RE-ADJUST”. Upon receiving streaming breathing rate data from the breathing rate sensor arrays, the LED screen will display numerical breathing rates as “12, 14, 16, 20, 24 BPM” (breaths per minute) in GREEN color along with audible alerts to indicate changes in breathing rate. If the dog's breathing rate rises or falls out of normal physiological ranges, the LED screen will display the breathing rate in YELLOW or RED to give warning, as well as produce an audible signal to alert the dog owner. Additionally, the dog's breathing rate data may be used by the harness system computer to generate status updates such as when rapid, shallow breathing patterns are measured, the update “PANTING” is displayed across the LED screen.

The harness system LED screen, or flat panel display, will also provide an activity level mode or display showing measurements collected by the pedometer, GPS module or other activity level sensor or modules. By manipulating the menu button or touch screen functionality, the dog owner may navigate through the user-interface (UX) of the harness system LED screen and arrive at the activity level menu. The activity level menu will display the dog's daily step-count data, distance walked, minutes of activity, minutes of rest, daily calories burned, and sleep time information. General activity status updates may be flashed or scrolled across the LED screen menu as ACTIVE, INACTIVE, or NORMAL to indicate overall daily activity levels for the dog and to assist the owner in providing health activity or exercise opportunities.

The harness system LED screen may furthermore provide emergency care notifications to the dog owner or veterinary provider in the case of health risk situations. For example, in a preferred embodiment of the harness system emergency care response, during temperature or body heat levels that rise to elevated levels, the LED screen may flash the notification “WATER” to alert the owner that the dog may be thirsty or de-hydrated. Alternatively, if the dog's temperature or body heat levels fall to below normal levels, the LED screen may flash the notification “HYPOTHERMIA” to alert the owner that the dog requires warmth and reduction to exposure. The harness system computer when measuring and interpreting a dramatic increase or decrease in body heat or temperature, coupled with elevated breathing rate levels consistent with panting, may issue the notification “SWEAT GLANDS” to alert the dog owner to check the sweat glands on the feet or the top of the dog's nose for the diagnosis of heat-stroke or other condition. Further embodiments of the proper care notifications may include updates to provide food, i.e., as the update “FOOD” is flashed across the LED screen. The harness system computer will track feeding habits of the owner's dog by receiving updates from the owner's mobile device or by tracking dog interactions with food bowl sensors or other unique sensor data signal traces or patterns to indicate when the dog has eaten. During the acquisition of vital sign data and activity level data, the harness system computer will upload and receive data from the cloud based system software. Health care data analytics may provide alerts or notifications to the harness system computer. For example, the system may determine that low activity levels coupled with specific vital sign measurements may indicate a healthcare issue, depression, or disease state with the owner's dog and the appropriate notification “VET” may be displayed on the LED screen, alerting the owner to take the dog to a proper animal care hospital or facility.

The harness system LED screen, or flat panel display, may furthermore provide dog mood status updates. The harness system computer will receive health data from the dog's vital signs and the harness mounted microphone to interpret the dog's audible bark sounds. This data may be processed with an algorithm to interpret which mood the dog is currently experiencing. For example in a preferred embodiment, the rapid increase in heart rate, breathing rate, the appearance of panting, or the measurement of specific audible sounds may be interpreted to the harness system computer as indicating the dog's increase in overall anxiety level and the appropriate notification will be displayed on the LED screen as “ANXIOUS”. Alternatively, the unique combination of currently measured vital signs, activity levels, and audile noises may result in the issuance of the appropriate updates indicating the dog's mood. For example in a preferred embodiment of the dog mood status notification, the dog's current daily activity level may be recorded as low or inactive. In combination with the sudden increase in activity level, the measurement of specific audible sounds, and an increase in heart rate and breathing rate may be interpreted by the harness system computer as the dog's mood being “PLAYFUL” and this update will be displayed on the LED screen. Furthermore, the place or location as indicated and recorded by the GPS module, of location based data, and past activity levels may be used by the harness system computer to generate mood status updates. During trips to the park for playful activity, the harness system computer will apply the GPS signal data to the determination and pattern recognition of appropriate mood updates as time and place are important factors in understanding a dog's mood.

The dog harness system disclosed here may be embodied in a full chest protector form factor with a surface area completely covering or covering most of the chest area of the dog. A preferred embodiment of the harness system is designed to wrap around the chest, sides and back of the dog's torso. The chest area of the harness will house the temperature sensors, heart rate sensors, breathing rate sensors, activity module electronics and related circuitry for wiring into the harness system on-board computer. The dog harness is designed with straps and webbing that can be opened from either side and allows quick connection and adjustment over the dog's torso and limbs. The harness may have labels for “SERVICE DOG” or the dog's name, i.e., “PRONTO”. Hooks are provided on the harness for leash attachments and for wheel chair attachments. Handles for the dog owner are provided on the top, left side, right side, or other harness locations in order to allow the owner to easily access the harness and control the dog from any side. An embodiment of the harness system may comprise four handles spaced at the back, left side, right side, and on top of the neck area of the harness. A light reflector is incorporated into the webbing or straps of the harness system in order to provided safety reflection when walking the dog at night. Anti-static material is used in the webbing to eliminate static shock from the dog's hair contacting and rubbing with the harness material. Inside the harness system between the dog and the harness is provided a honeycomb padding structure material to give a swaddled and secure and comfortable feeling for the dog. Nylon webbing for the harness is secured with plastic side-release or center release buckles. Velcro straps are added as a fail-safe mechanism over the plastic buckles and provides a more secure method of attaching the harness system to the dog. If a buckle becomes released or fails, the velcro straps serve as a back-up method of keeping the harness securely attached to the dog.

1. A dog harness system for health data collection and monitoring comprising:
a plurality of harness integrated temperature sensors for measuring body heat;
a plurality of harness integrated transducer sensors for measuring heart rate;
a plurality of harness integrated transducer sensors for measuring breathing rate; and
a pedometer or GPS module for measuring the dog's activity level;
wherein the temperature sensors, transducer sensors, and pedometer or GPS module are connected to the harness system computer and flat panel display screen for data processing, diagnosis, and providing live health data notifications, vital sign information, and activity level updates.

2. The dog harness system of claim 1, wherein the plurality of temperature sensors are mounted in a plastic frame housing the sensor probe rounded metallic tip, a force plate, retaining rings, and a spring force tensioner producing a positive downward force on the sensor probe tip, wherein the dampening and spring force may be adjusted with a thumb screw.

3. The dog harness system of claim 1, wherein the plurality of temperature sensors have a rounded metallic probe tip with harness strap activated and adjustable tension to develop downward force on the sensor probe tip to a hairless part of the dog's chest or torso.

4. The dog harness system of claim 1, wherein the plurality of temperature sensors comprising rounded metallic tips are aligned along the chest facing area of the harness and positive tension towards the dog's body is produced by curved plastic ribbing embedded inside the harness system in the chest area and adjustment of the harness presses the sensor probe tips against the dog's chest.

5. The dog harness system of claim 1, wherein the flat panel display screen provides a user-interface (UX) with menu functionality for each sensor signal data modality, wherein the temperature sensor probe menu comprises numerical or graphical data to alert the dog owner for healthy and normal physiological temperatures, transitions out of safe temperatures, and warnings for hyperthermic or hypothermic conditions; and wherein the data is displayed constantly, flashed periodically during set intervals, or alternatively flashed on the screen during changes from currently measured signal levels.

6. The dog harness system of claim 1, wherein the dog's health data, vital signs and activity levels are uploaded to cloud based services for data analytics across customer data populations, automated report generation, and transmission to veterinary professionals for diagnosis, treatment recommendation and provision of emergency care and wherein emergency care notifications are flashed upon the flat panel display screen to alert the dog owner to potential healthcare issues and recommended care actions.

7. The dog harness system of claim 1, wherein the harness system attaches by ring to a leash and moves the center of gravity of the leash pulling forces away from the neck or collar area and towards the middle of the dog's body and evenly distributes the leash tugging and pulling forces along the dog's torso and stomach and thereby avoids injury and damage to the dog's neck, throat, hips and joints.

8. A dog harness system for health data collection and monitoring comprising:
a plurality of temperature sensors, heart rate sensors, and breathing rate sensors for measuring the dog's vital signs;
a pedometer module and GPS module for measuring the dog's activity levels;
a microphone for sampling the dog's audible signals;
a harness system computer for processing and sampling signal data from the sensors, activity level modules and microphone; and
wherein the harness system computer displays health data and vital sign information on a harness mounted flat panel display with menu functionality, status updates, and emergency care notifications provided as feedback from measured and collected signal data.

9. The dog harness system of claim 8, wherein the plurality of temperature sensors are mounted in a plastic frame housing the sensor probe rounded metallic tip, a force plate, retaining rings, and a spring force tensioner producing a positive downward force on the sensor probe tip, wherein the dampening and spring force may be adjusted with a thumb screw.

10. The dog harness system of claim 8, wherein the plurality of temperature sensors have a rounded metallic probe tip with harness strap activated and adjustable tension to develop downward force on the sensor probe tip to a hairless part of the dog's chest or torso.

11. The dog harness system of claim 8, wherein the plurality of temperature sensors comprising rounded metallic tips are aligned along the chest facing area of the harness and positive tension towards the dog's body is produced by curved plastic ribbing embedded inside the harness system in the chest area and adjustment of the harness presses the sensor probe tips against the dog's chest.

12. The dog harness system of claim 8, wherein the flat panel display screen provides a user-interface (UX) with menu functionality for each sensor signal data modality; wherein the temperature sensor probe menu comprises numerical or graphical data to alert the dog owner for healthy and normal physiological temperatures, transitions out of safe temperatures, and warnings for hyperthermic or hypothermic conditions; and wherein the data is displayed constantly, flashed periodically during set intervals, or alternatively flashed on the screen during changes from currently measured signal levels.

13. The dog harness system of claim 8, wherein the dog's health data, vital signs and activity levels are uploaded to cloud based services for data analytics across customer data populations, automated report generation, and transmission to veterinary professionals for diagnosis, treatment recommendation and provision of emergency care and wherein emergency care notifications are flashed upon the flat panel display screen to alert the dog owner to potential healthcare issues and recommended care actions.

14. The dog harness system of claim 8, wherein the harness system attaches by ring to a leash and moves the center of gravity of the leash pulling forces away from the neck or collar area and towards the middle of the dog's body and evenly distributes the leash tugging and pulling forces along the dog's torso and stomach and thereby avoids injury and damage to the dog's neck, throat, hips and joints.

15. A dog harness system for health data collection and monitoring comprising:
a plurality of temperature sensors, heart rate sensors, and breathing rate sensors for measuring the dog's vital signs;
a pedometer module and GPS module for measuring the dog's activity levels;
a microphone for sampling the dog's audible signals;
a harness system computer for processing and sampling signal data from the sensors, activity level modules and microphone and uploading the measured signal data to cloud-based services for customer data analytics and processing; and
wherein the sensors, activity modules, microphone and harness system computer are embedded into a full chest protector form factor covering the chest area of the dog; the harness is attached with straps, webbing, plastic buckles, velcro, and a honeycomb structure inner surface for comfortably swaddling the dog; and wherein the harness system computer displays health data and vital sign information on a harness mounted flat panel display screen with menu functionality, status updates, and emergency care notifications provided as feedback from measured and collected signal data and from cloud based services.

16. The dog harness system of claim 15, wherein the plurality of temperature sensors are mounted in a plastic frame housing the sensor probe rounded metallic tip, a force plate, retaining rings, and a spring force tensioner producing a positive downward force on the sensor probe tip, wherein the dampening and spring force may be adjusted with a thumb screw.

17. The dog harness system of claim 15, wherein the plurality of temperature sensors have a rounded metallic probe tip with harness strap activated and adjustable tension to develop downward force on the sensor probe tip to a hairless part of the dog's chest or torso.

18. The dog harness system of claim 15, wherein the plurality of temperature sensors comprising rounded metallic tips are aligned along the chest facing area of the harness and positive tension towards the dog's body is produced by curved plastic ribbing embedded inside the harness system in the chest area and adjustment of the harness presses the sensor probe tips against the dog's chest.

19. The dog harness system of claim 15, wherein the flat panel display screen provides a user-interface (UX) with menu functionality for each sensor signal data modality; wherein the temperature sensor probe menu comprises numerical or graphical data to alert the dog owner for healthy and normal physiological temperatures, transitions out of safe temperatures, and warnings for hyperthermic or hypothermic conditions; and wherein the data is displayed constantly, flashed periodically during set intervals, or alternatively flashed on the screen during changes from currently measured signal levels.

20. The dog harness system of claim 15, wherein the dog's health data, vital signs and activity levels are uploaded to cloud based services for data analytics across customer data populations, automated report generation, and transmission to veterinary professionals for diagnosis, treatment recommendation and provision of emergency care and wherein emergency care notifications are flashed upon the flat panel display screen to alert the dog owner to potential healthcare issues and recommended care actions.
